(19) United States
(12) Patent Application Publication
(10) Pub. No.: US 2023/0348531 A1
(43) Pub. Date: Nov. 2, 2023

(54) ISOLATION AND PURIFICATION OF BACTERIOCIN PRODUCED BY LACTOBACILLUS SAKEI AND ANTIBACTERIAL USE THEREOF AND LACTIC ACID BACTERIA USED

(71) Applicant: Zhejiang Gongshang University, Hangzhou (CN)

(72) Inventors: Qing Gu, Hangzhou (CN); Pengxin Ye, Hangzhou (CN); Ping Li, Hangzhou (CN); Qingqing Zhou, Hangzhou (CN); Huifei Shentu, Hangzhou (CN)

(21) Appl. No.: 18/054,144

(22) Filed: Nov. 10, 2022

(30) Foreign Application Priority Data
Apr. 30, 2022 (CN) 202210476564.7

(51) Int. Cl. C07K 1/36; B01D 15/36; C07K 1/34

(52) U.S. Cl. CPC C07K 1/36; B01D 15/362; C07K 1/34

(57) ABSTRACT

The present disclosure discloses Lactobacillus sakei ZFM225, with a preservation number of CCTCC NO:M 2016669. The present disclosure further discloses an isolation and purification method of an Lactobacillus sakei ZFM225 bacteriocin, including the steps of: precipitating fermentation supernatant of the Lactobacillus sakei ZFM225 by an ammonium sulfate precipitation method to obtain crude protein; desalting the crude protein to obtain crude protein extract; isolating the crude protein extract by cation exchange chromatography, performing elution with a gradient eluent containing 0.5 M to 1.0 M NaCl, and desalting the obtained eluate to obtain a preliminary protein purified solution; and then purifying the preliminary protein purified solution by reversed-phase high-performance liquid chromatography to obtain the Lactobacillus sakei ZFM225 bacteriocin. The Lactobacillus sakei ZFM225 bacteriocin has antibacterial activity on both gram-positive bacteria and gram-negative bacteria.

### CROSS-REFERENCE TO RELATED APPLICATIONS

The present application claims the benefit of Chinese Patent Application No. 202210476564.7 filed on Apr. 30, 2022, the contents of which are incorporated by reference herein in its entirety.

### TECHNICAL FIELD

The present disclosure belongs to the technical field of food biotechnology, and specifically relates to isolation and purification of a bacteriocin produced by Lactobacillus sakei and an antibacterial use thereof and lactic acid bacteria used.

### BACKGROUND

Food spoilage can be seen everywhere in our daily life, especially for foods with relatively high water content, such as fish, meat, vegetables, etc., which usually deteriorate in a very short time and cannot be eaten, thus causing great waste. Generally, the causes for the food spoilage are as follows: first, the microbial action, food contamination caused by food-borne such as Stappathogens Hylococcus aureus and Bacillus subtilis; second, the enzymes in food; third, the humidity and temperature of the air. While microbial contamination is the main cause of the food spoilage. Although emergence of a food preservative can effectively alleviate the food spoilage problem caused by microorganisms, with the improvement of human living standards and medical standards, it is found that many food additives used today, especially chemical additives, have a risk of carcinogenesis, teratogenesis and even mutation if they are consumed for a long time. Therefore, development of a novel non-toxic and harmless food preservative has become a focus of research in the food field.

Lactic acid bacteria are generally recognized as non-toxic microorganisms and can be used in food. The lactic acid bacteria have been used as a starter to produce fermented products and some strains with a probiotic function have been used for producing probiotic products. The vast majority of lactic acid bacteria can produce a variety of inhibitory substances, among which bacteriocin is a protein or polypeptide with an inhibitory effect that is produced by ribosome, which has attracted extensive attention due to its advantages such as no toxicity, no residue and no drug resistance. At present, only Nisin is a bacteriocin allowed to be used as a food additive worldwide. Since the Nisin can only inhibit gram-positive pathogens and has no inhibitory effect on gram-negative bacteria such as Escherichia coli, its application is limited, so it is urgent to find new bacteriocin with broad-spectrum inhibitory effect.

### SUMMARY

The problem to be solved by the present disclosure is to provide a Lactobacillus sakei bacteriocin obtained by isolation and purification, an antibacterial use thereof and lactic acid bacteria used.

To solve the above technical problems, the present disclosure provides an isolation and purification method of Lactobacillus sakei ZFM225 bacteriocin, which includes the steps of:
1) preparation of fermentation supernatant of Lactobacillus sakei ZFM225:
inoculating the Lactobacillus sakei ZFM225 into an MRS liquid medium for fermentation, and centrifuging obtained fermentation broth to obtain the fermentation supernatant;
2) precipitating the fermentation supernatant by an ammonium sulfate precipitation method to obtain crude protein, and desalting the crude protein to obtain crude protein extract;
3) isolating the crude protein extract by cation exchange chromatography, performing elution with a gradient eluent containing 0.5 M to 1.0 M NaCl, and desalting an obtained eluate to obtain a preliminary protein purified solution;
purifying the protein preliminary purified solution by reversed-phase high-performance liquid chromatography to obtain the Lactobacillus sakei ZFM225 bacteriocin.

As an improvement of the isolation and purification method of the Lactobacillus sakei ZFM225 bacteriocin of the present disclosure, the step 1) is as follows:
the Lactobacillus sakei ZFM225 cultured to a logarithmic growth phase is inoculated with an inoculation amount of 2% (v/v) into the MRS liquid medium with pH 6.5, and is cultured at 37° C. for 24 h; the obtained fermentation broth is centrifuged (at 8000 r/min and 4° C. for 30 min) to obtain the fermentation supernatant.

As a further improvement of the isolation and purification method of the Lactobacillus sakei ZFM225 bacteriocin of the present disclosure, the step 2) is as follows: Ammonium sulfate powder is added to the fermentation supernatant until saturation is 70±2%, stirring is performed at 4±1° C. for 10 to 14 h, and collected precipitate is centrifuged to obtain the crude protein;
the crude protein is desalted by using a sephadex column G-10 (Φ1.6×50) to obtain the crude protein extract.

As a further improvement of the isolation and purification method of the Lactobacillus sakei ZFM225 bacteriocin of the present disclosure, the step 3) is as follows:
a cation exchange chromatographic column (HiPrepTMSP XL 16/10, GE Healthcare) is adopted for the crude protein extract, the concentration of sodium chloride in the gradient eluent is increased from 0 M to 1 M at a constant rate within 30 min, and a corresponding eluate of 0.5 M to 1 M NaCl gradient eluent is collected; desalination is performed by using the sephadex column G-10 to obtain the protein preliminary purified solution;
in gradient elution, use a mixture of a buffer solution and an eluent as a gradient eluent;
the buffer solution is obtained from 30 mM sodium acetate by adjusting pH to 4, and performing 0.22 μm suction filtration, and ultrasonic treatment for 20 min;
the eluent is obtained from 30 mM sodium acetate and 1 M sodium chloride by adjusting pH to 4, and performing 0.22 μm suction filtration and ultrasonic treatment for 20 min.

As a further improvement of the isolation and purification method of the Lactobacillus sakei ZFM225 bacteriocin of the present disclosure, the step 3) is as follows:
the protein preliminary purified solution is further purified by preparative C18 reversed-phase high-performance liquid chromatography;
a mobile phase A is ultra-pure water containing 0.05% (v/v) TFA, and a mobile phase B is acetonitrile containing 0.05% TFA (v/v);
that is, TFA accounting for 0.05% of the volume content of the ultra-pure water is added into the ultra-pure water to serve as the mobile phase A, and TFA accounting for 0.05% of the volume content of the acetonitrile is added into the acetonitrile to serve as the mobile phase B;
TFA is a trifluoroacetic acid.

Mobile Phase Elution Gradient

| | Flow | | |
|---|---|---|---|
| Time | rate | Mobile | Mobile |
| (min) | (mL/min) | phase A | phase B |
| 0 | 5 | 95 | 5 |
| 30 | 5 | 5 | 95 |
| 50 | 5 | 100 | 0 |

A corresponding collected eluate of 34.5% to 37.5% mobile phase B elution is collected to obtain a Lactobacillus sakei ZFM225 bacteriocin solution.

The present disclosure further provides a use of the Lactobacillus sakei ZFM225 bacteriocin solution: the bacteriocin solution has inhibitory activity on both gram-positive bacteria and gram-negative bacteria.

The gram-positive bacteria include Micrococcus luteus 10209, Staphylococcus aureus D48, Staphylococcus muscae, Staphylococcus carnosus pCA 44, and Staphylococcus carnosus pet 20.

The gram-negative bacteria include Escherichia coli DH5 α, Salmonella paratyphi A CMCC 50093, Salmonella choleraesuis ATCC 13312, and Pseudomonas aeruginosa ATCC 47085.

The present disclosure is specific as follows:
(1) An object of the present disclosure is to provide screening of a strain of high-quality lactic acid bacterium with a broad-spectrum antibacterial effect, which includes: Lactic acid bacteria are isolated from fresh milk through a calcium dissolving zone and colony morphology observation, and their inhibitory activity are tested by an Oxford cup agar diffusion method to screen out a strain of lactic acid bacterium with an inhibitory effect and highest inhibitory activity on both the gram-positive bacteria and the gram-negative bacteria. In combination with morphological identification, physiological and biochemical identification and 16S rDNA homology analysis, it is identified as Lactobacillus sakei, which is named the Lactobacillus sakei ZFM225.

Preservation Name: The Lactobacillus sakei ZFM225, Preservation Authority: China Center for Type Culture Collection, Preservation Address: Wuhan University, Wuhan, China, Preservation No.: CCTCC NO:M 2016669, and Preservation Date: Nov. 23, 2016.
(2) An object of the present disclosure is to provide an isolation and purification method of Lactobacillus sakei ZFM225 bacteriocin, which includes: The fermentation broth of the Lactobacillus sakei ZFM225 is centrifuged to obtain the fermentation supernatant. The fermentation supernatant is precipitated stepwise by using a saturated ammonium sulfate precipitation method, and a 70% ammonium sulfate precipitation solution has the inhibitory activity, thus obtaining crude protein extract; the crude protein extract is isolated by the anion exchange chromatography, and elution is performed with a 0.5 M to 1.0 M NaCl solution, thus obtaining the preliminary protein purified solution; then, the preliminary protein purified solution is purified by the reversed-phase high-performance liquid chromatography, and elution is performed with a 34.5% to 37.5% acetonitrile solution to obtain the Lactobacillus sakei ZFM225 bacteriocin solution.
(3) The optimal culture conditions of the fermentation broth of the Lactobacillus sakei ZFM225 are as follows: The Lactobacillus sakei ZFM225 is inoculated with an inoculation amount of 2% (v/v) into an MRS liquid medium with pH 6.5, and is cultured at 37° C. for 24 h. Under this condition, the potency of the fermentation supernatant is 266 IU/mL, which is increased by 1.8 times in comparison with 146 IU/mL before optimization.
(4) The Lactobacillus sakei ZFM225 bacteriocin obtained by the present disclosure is detected as a single peak by analytical HPLC, indicating that relatively good purification is realized.
(5) The Lactobacillus sakei ZFM225 bacteriocin obtained by the present disclosure has a molecular weight of about 14 kDa, a protein concentration of 4.85 mg/L, specific activity of 808 IU/mg, and a purification fold of 75.94 times.
(6) The Lactobacillus sakei ZFM225 bacteriocin obtained by the present disclosure has an antibacterial use and a relatively broad inhibitory spectrum, and can effectively inhibit the gram-positive bacteria and the gram-negative bacteria (such as Escherichia coli and Salmonella), among them, the inhibitory ability to Micrococcus luteus and Staphylococcus aureus is the highest.
(7) The Lactobacillus sakei ZFM225 bacteriocin obtained by the present disclosure has high stability. The activity of the bacteriocin is basically stable after being treated at 100° C. for 30 min. The bacteriocin has relatively high inhibitory activity in an acidic condition, but has decreased significantly activity in an alkaline condition. The bacteriocin is sensitive to protease, especially after trypsin and pepsin treatment, its activity is almost completely lost, which can make it enter the body and be degraded into small molecules to reduce residues.

Compared with the prior art, the present disclosure has the following advantages.
1. The present disclosure obtains the Lactobacillus sakei ZFM225 bacteriocin from the fermentation supernatant of the Lactobacillus sakei ZFM225 by applying a three-step method of “ammonium sulfate precipitation-cation exchange chromatography-reversed-phase high-performance liquid chromatography”, which has a broad-spectrum antibacterial property and thermal stability.
2. The present disclosure optimizes the fermentation conditions for high yield of the Lactobacillus sakei ZFM225 bacteriocin, so that its potency is increased by 1.8 times.

To sum up, the present disclosure obtains the novel Lactobacillus sakei ZFM225 bacteriocin by establishing an isolation and purification technology, and determines broad-spectrum antibacterial characteristics, thermal stability, and enzyme sensitivity of the bacteriocin, which has a potential to be applied to food preservation.

### BRIEF DESCRIPTION OF THE DRAWINGS

Specific implementations of the embodiments of the present disclosure are described in further detail in the following with reference to the accompanying drawings.

FIG. 1A to FIG. 1D show screening of lactic acid bacteria producing bacteriocin with a broad-spectrum inhibitory effect, wherein:

FIG. 1A is a strain growth state of lactic acid bacteria in an isolation medium;

FIG. 1B is eliminating an influence of an organic acid on inhibitory activity, where sign 1 is fermentation supernatant of a strain 03, sign 2 is fermentation supernatant adjusted to pH 5, sign 3 is a hydrochloric acid MRS medium with pH 5, sign 4 is an acetic acid MRS medium with pH 5, and sign 5 is a lactic acid MRS medium with pH 5;

FIG. 1C is a hydrogen peroxide elimination experiment, where sign 1 is fermentation supernatant; sign 2 is fermentation supernatant obtained after treatment with a hydrogen oxide enzyme for 2 h; and

FIG. 1D is an effect of different protease treatments on inhibitory activity of the strain 03.

FIG. 2A to FIG. 2C show identification of bacteriocins producing lactic acid bacteria, wherein:

FIG. 2A is a colony morphology of Lactobacillus sakei ZFM225, FIG. 2B is a Gram staining assay of the Lactobacillus sakei ZFM225, and FIG. 2C is a phylogenetic tree of a 16S rDNA gene sequence system of the Lactobacillus sakei ZFM225.

FIG. 3A to FIG. 3E show purification and identification of bacteriocins, wherein:

FIG. 3A is an SP-Sepharose purification chromatogram, where except a marked elution peak, the rest are flowthrough peaks; FIG. 3B is inhibitory activity of a cation exchange chromatography component; FIG. 3C is a preparative HPLC purification chromatogram; FIG. 3D is analytical high performance liquid chromatography; and FIG. 3E is an SDS-PAGE map of an Lactobacillus sakei ZFM225 bacteriocin, where sign M is a protein Marker, and sign 1 is a purified Lactobacillus sakei ZFM225 bacteriocin.

FIG. 4 shows a minimum inhibitory concentration of a bacteriocin ZFM225.

FIG. 5 shows thermal stability of an Lactobacillus sakei ZFM225 bacteriocin.

FIG. 6 shows an effect of different pH values on inhibitory activity of an Lactobacillus sakei ZFM225 bacteriocin.

FIG. 7 shows an effect of different protease treatments on inhibitory activity of a Lactobacillus sakei ZFM225 bacteriocin.

### DETAILED DESCRIPTION

The present disclosure is further described below with reference to specific embodiments, and the advantages and characteristics of the present disclosure become more apparent with the description. However, these examples are only exemplary and do not constitute any limit to the scope of the present disclosure.

### Embodiment 1: Screening of Lactic Acid Bacteria Producing Bacteriocin with Broad-Spectrum Inhibitory Effect

(1) Screening of Lactic Acid Bacteria from Raw Milk Samples

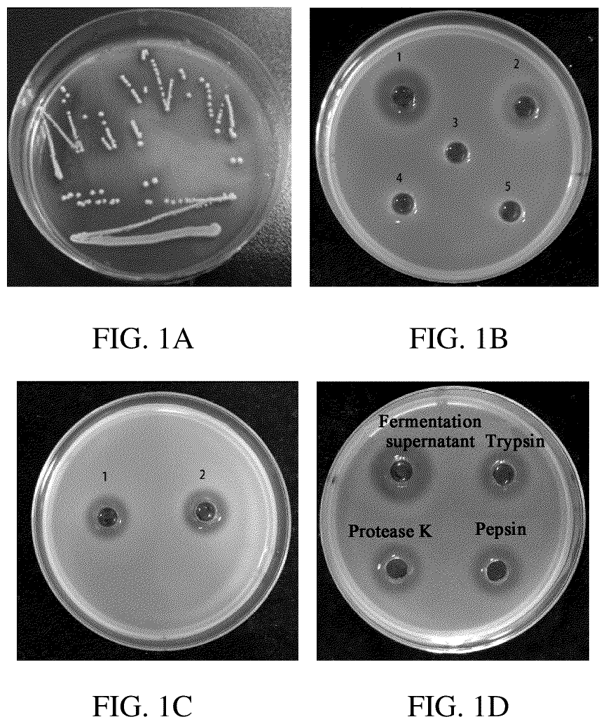

Isolated samples came from raw milk of the Hangzhou dairy farm. After collection, the isolated samples were gradient-diluted tenfold with normal saline. For each gradient, 100 μL of the isolated sample was taken and spread onto plates of a lactic acid bacteria screening medium (MRS medium containing 2% calcium carbonate). The isolated sample was spread onto three plates for each gradient, and is cultured upside down at 37° C. for 48 h. Single colonies with an obvious calcium dissolving zone were selected and inoculated into a 10 mL MRS liquid medium for activation. Then a bacterial solution was spread onto the MRS screening medium for streaking, which was repeated for multiple times, to obtain single colonies with the obvious calcium dissolving zone. These single colonies were picked out for Gram staining and catalase test. The single colonies preliminarily identified as lactic acid bacteria were inoculated into the MRS medium, cultured at 37° C. for 24 h, activated and numbered, and preserved at −80° C. Lactic acid bacteria that may produce the calcium dissolving zone were screened out. As shown in FIG. 1A, 30 single colonies with the obvious calcium dissolving zone were selected. Inhibitory activity analysis was performed on the 30 isolated lactic acid bacteria by taking the gram-positive bacteria, Micrococcus luteus 10209 and the gram-negative bacteria, Escherichia coli DH5α as indicator bacteria, to obtain 12 strains with relatively high inhibitory activity. The obtained strains were numbered 01, 02, 03, 04, 05, 06, 07, 08, 09, 10, 11, 12.

(2) Preliminary Screening of Lactic Acid Bacteria with Inhibitory Effect

Preparation of corresponding fermentation supernatant from strains 01 to 12: A loopful of strains was taken and added to a 10 mL MRS medium, and was cultured at 30° C. for 24 h. After the culture, the cultured strains were centrifuged at 8000 r/min at 4° C. for 30 min. Thalli were discarded. Supernatant substances were filtered with a 0.22 μM filter membrane to remove impurities, to obtain corresponding fermentation supernatant.

The inhibitory activity of the fermentation supernatant was tested by using an Oxford cup agar diffusion method, and a strain with a best inhibitory effect was selected for subsequent experiments. In a biosafety cabinet, the indicator bacteria were added with an inoculation amount of 1% (v/v) into an LB semi-solid medium that had been fully heated and melted and kept at a constant temperature of 55° C. After being shaken and mixed well, the indicator bacteria were poured into a disposable culture dish that had been evenly placed with sterile Oxford cups. The Oxford cups were carefully pulled out after the mixture was fully cooled and solidified, and 100 μL of each fermentation supernatant to be tested was added into all wells. A dish was allowed to stand for half an hour at 4° C. to make the fermentation supernatant fully diffuse, and then was put in an incubator for culture for 12 h. An inhibitory zone diameter was measured with a vernier caliper and its transparency was observed. Three parallels were made on the fermentation supernatant of each strain.

The inhibitory activity of the 12 lactic acid bacteria numbered 01 to 12 obtained in step (1) on the Micrococcus luteus 10209 and the Escherichia coli DH5α is shown in Table 1, and it was analyzed from results that the strain 03 had a best inhibitory effect on the two indicator bacteria, and therefore, the strain 0.3 was selected for the subsequent experiments.

TABLE 1
Inhibitory Effect of Lactic Acid Bacteria Fermentation
Supernatant on Indicator Bacteria

| | Inhibitory Zone | Inhibitory Zone |
|---|---|---|
| | Diameter (mm) of | Diameter (mm) of |
| Strain | Micrococcus luteus | Escherichia coli |
| Name | 10209 | DH5α |
| 01 | 14.32 ± 0.78 | 15.21 ± 0.45 |
| 02 | 15.03 ± 0.13 | 14.57 ± 0.24 |
| 03 | 16.71 ± 0.88 | 16.01 ± 0.52 |
| 04 | 16.13 ± 0.32 | 15.78 ± 0.23 |
| 05 | 13.91 ± 0.55 | 12.56 ± 0.65 |
| 06 | 15.32 ± 0.47 | 14.41 ± 0.13 |
| 07 | 15.27 ± 0.43 | 14.67 ± 0.32 |
| 08 | 16.34 ± 0.43 | 13.92 ± 0.46 |
| 09 | 15.55 ± 0.98 | 16.21 ± 0.33 |
| 10 | 14.82 ± 0.78 | 16.73 ± 0.54 |
| 11 | 13.92 ± 0.38 | 14.65 ± 0.32 |
| 12 | 16.12 ± 0.47 | 15.91 ± 0.23 |

(3) Re-Screening of Bacteriocin Producing Lactic Acid Bacteria

In addition to bacteriocin-like inhibitory substances, lactic acid bacteria may also produce substances with the inhibitory activity, such as an organic acid and hydrogen peroxide in their fermentation process. Therefore, to determine that it was the bacteriocin to have the broad-spectrum inhibitory effect in the lactic acid bacteria, it was necessary to eliminate an inhibitory interference effect of the organic acid and the hydrogen peroxide on the indicator bacteria. In addition, lactic acid bacteria bacteriocin was a protein substance produced by the lactic acid bacteria in their fermentation process. Therefore, the fermentation supernatant may be treated with protease to see a change of the inhibitory activity, so as to determine whether there was a protein inhibitory substance or not.

1) Elimination of Interference of Organic Acid

The pH of the fermentation supernatant was adjusted to 5 with 1 M NaOH. The pH of the MRS liquid medium was adjusted to the same pH with a lactic acid and an acetic acid. Taking the original fermentation supernatant (pH 4.23) as control, and taking the Micrococcus luteus 10209 as the indicator bacteria, its inhibitory activity was determined by using the above Oxford cup agar diffusion method. The bacteriostasis experiment results were shown in FIG. 1B. It was found from the results that the inhibitory effect of the strain 03 on the indicator bacteria was weakened after the interference of the organic acid was eliminated, indicating that the organic acid produced by the strain 03 played a role in its inhibitory ability. However, after the influence of the organic acid was eliminated, the fermentation supernatant still had certain inhibitory activity, indicating that the inhibitory effect of the strain 03 on the indicator bacteria was not only caused by the organic acid, and there were still other substances that have the inhibitory effect on the indicator bacteria.

2) Elimination of Interference of Hydrogen Peroxide

10 mL of fermentation supernatant (process parameters of concentration: 37° C. and vacuum) was concentrated with a rotary evaporator to obtain 5 mL of concentrated fermentation supernatant.

A catalase working solution with a concentration of 2.5 to 5.0 mg/mL was prepared. The concentrated fermentation supernatant was made to be mixed with the catalase working solution in a ratio of 1:1. Taking the fermentation supernatant with no catalase added as blank control, and the mixture was treated in a water bath at 37° C. for 2 h. Its inhibitory activity was determined with the Oxford cup agar diffusion method and their differences were compared. The results were as shown in FIG. 1C. After treatment with a hydrogen oxide enzyme for 2 h, the inhibitory activity and the fermentation supernatant basically had no change. The hydrogen peroxide had inhibitory activity, and the catalase may promote decomposition of the hydrogen peroxide into molecular oxygen and water. This experiment showed that the inhibitory activity in the fermentation supernatant was contributed by substances other than the hydrogen peroxide.

3) Determination of Bacteriocin Producing Substances

Enzyme solutions of proteinase K, trypsin and pepsin were prepared, 5 mg/mL for each enzyme solution. The pH of the fermentation supernatant was adjusted to optimum pH of each protease. Each corresponding enzyme solution was added to make its final concentration 1 mg/mL, and was treated in a water bath at 37° C. for 2 h. The pH was adjusted to the original pH. Taking the fermentation supernatant without enzyme treatment as the blank control, bacteriostasis experiments were conducted, and their differences were compared. The results were shown in FIG. 1D. It was found that the inhibitory zone diameter after the treatment of three proteases was decreased to a certain extent, indicating that an inhibitory substance contained in the fermentation supernatant of the strain 03 was sensitive to the protease. From this result, it may be preliminarily determined that the fermentation supernatant of the strain 03 contained a protein or polypeptide-like substance with certain inhibitory activity.

(4) Identification of Bacteriocin Producing Lactic Acid Bacteria

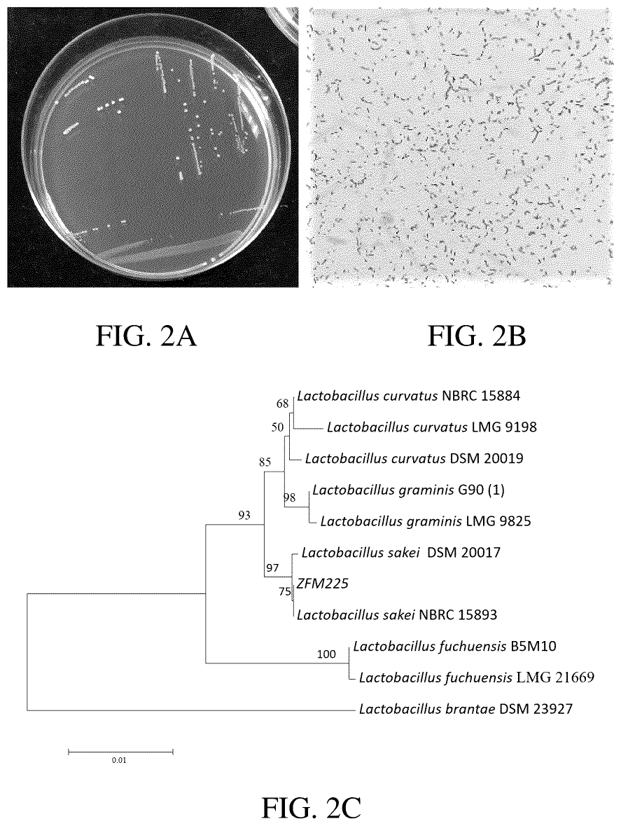

As shown in FIG. 2A, the single colony was white and round, with neat edges and a smooth surface, and the size between 0.5 to 1 mm. As shown in FIG. 2B, gram staining results showed purple positive bacteria, and microscopic examination results showed that the rod-shaped was consistent with Lactobacillus in terms of characteristics. As shown in FIG. 2C, through further 16S rDNA gene-based molecular biological identification, the strain 03 was identified as Lactobacillus sakei, and was formally named the Lactobacillus sakei ZFM225. The preservation information of this strain was as follows:

Preservation Name: The Lactobacillus sakei ZFM225, Preservation Authority: China Center for Type Culture Collection, Preservation Address: Wuhan University, Wuhan, China, Preservation No.: CCTCC NO:M 2016669, and Preservation Date: Nov. 23, 2016.

### Embodiment 2: Optimization of Fermentation Conditions for Bacteriocin Produced by Lactobacillus sakei ZFM225

The following indicator bacteria used for determination of the inhibitory activity were all the Micrococcus luteus 10209.

(1) Influence of Culture Temperature

The Lactobacillus sakei ZFM225 was inoculated with an inoculation amount of 1% (v/v) into the MRS liquid medium, and underwent stationary culture at 25° C., 30° C., 37° C. and 45° C. for 24 h. Its OD₆₀₀ value and inhibitory activity (expressed by the inhibitory zone diameter) were determined. The results showed that optimum fermentation temperature was 37° C. Under this condition, the inhibitory zone diameter was maximum, which was 16.70 mm.

(2) Influence of Culture Time

The Lactobacillus sakei ZFM225 was inoculated with an inoculation amount of 1% (v/v) into the MRS liquid medium, with culture temperature of 30° C. Samples were taken at time points of 0, 2, 4, 6, 8, 10, 14, 18, 24, 30, 36, 42, and 48 h of the culture time. Their OD values and inhibitory activity (expressed by the inhibitory zone diameter) were determined. It was determined that optimum fermentation time was 24 h. At this time, the inhibitory zone diameter was maximum, which was 16.04 mm.

(3) Influence of Inoculation Amount

The Lactobacillus sakei ZFM225 was inoculated with an inoculation amount of 1%, 1.5%, 2%, 2.5% and 3% (v/v) into the MRS liquid medium, and underwent stationary culture at 37° C. for 24 h. Its OD value and inhibitory activity (expressed by the inhibitory zone diameter) were determined. It was determined that optimum inoculation amount was 2%. At this time, the inhibitory zone diameter was maximum, which was 16.47 mm.

(4) Influence of Initial pH

The initial pH of the MRS liquid medium was adjusted to 4.5, 5.0, 5.5, 6.0, 6.5, 7.0, 7.5, 8.0 and 8.5. The Lactobacillus sakei ZFM225 was inoculated with an inoculation amount of 2% (v/v) into the MRS liquid medium adjusted to different pH values, and underwent stationary culture at 37° C. for 24 h. Its OD value and inhibitory activity (expressed by the inhibitory zone diameter) were determined. It was determined that optimum initial pH was 6.5. At this time, the inhibitory zone diameter was maximum, which was 16.77 mm.

It was finally determined that optimal culture conditions of the bacteriocin produced by the Lactobacillus sakei ZFM225 were: the bacteriocin was inoculated with the inoculation amount of 2% (v/v) into the MRS liquid medium with pH 6.5, and underwent stationary culture at 37° C. for 24 h. Under this condition, the potency of the fermentation supernatant was 266 IU/mL, which was increased by 1.8 times in comparison with 146 IU/mL before optimization.

Note: Parameters before optimization: The inoculation amount was 1% (v/v), the pH of the MRS liquid medium was 7, and stationary culture was performed at 30° C. for 24 h.

### Embodiment 3: Isolation and Purification of Lactobacillus sakei ZFM225 Bacteriocin

(1) Preparation of Fermentation Supernatant of Lactobacillus sakei ZFM225

The Lactobacillus sakei ZFM225, preserved at −80° C., streaked onto an MRS solid medium plate, and cultured at 37° C. After the single colonies grew out, single colonies were picked out therefrom and put in the 10 mL MRS liquid medium. The picked out single colonies were cultured to their growth logarithmic phases. The cultured single colonies were inoculated with an inoculation amount of 2% (v/v) into the 20 mL MRS liquid medium with pH 6.5, and were subcultured to its logarithmic phase to obtain an inoculum. Before use, the thalli concentration of the inoculum was adjusted to OD₆₀₀=0.6 with the normal saline. The inoculum was inoculated by the optimal culture condition, the inoculation amount of 2% (v/v), into the 1 L MRS liquid medium with pH 6.5, and was cultured at 37° C. for 24 h. The obtained fermentation broth was centrifuged at 8000 r/min and 4° C. for 30 min to obtain the fermentation supernatant (lactic acid bacteria fermentation supernatant), which was placed at 4° C. for later use.

(2) Ammonium Sulfate Precipitation

1 L of the lactic acid bacteria fermentation supernatant was taken, and ammonium sulfate powder was slowly added until saturation was 20%, 30%, 40%, 50%, 60%, 70%, 80%, 90% and 100% (at 25° C., ammonium sulfate powder saturation was 707 g/L). The mixture was placed in a chromatography cabinet at 4° C. and stirred overnight, and was centrifuged at 8000 r/min and 4° C. for 30 min and precipitate was collected. The precipitate was dissolved in deionized water (the concentration was 5.56 mg/mL) to obtain a protein salt solution. The activity was detected by the Oxford cup agar diffusion method. The indicator bacterium was the Micrococcus luteus 10209. The results showed that crude protein precipitated at 70% ammonium sulfate concentration had the best inhibitory effect, and the inhibitory zone diameter was 15.45 mm. Therefore, crude protein extract was obtained for subsequent experiments after the crude protein precipitated at the 70% ammonium sulfate concentration was desalted by using the sephadex column G-10 (Φ1.6×50).

The 70% ammonium sulfate precipitation: about 500 g ammonium sulfate powder was added to the 1 L of lactic acid bacteria fermentation supernatant.

The desalination was carried out by using the sephadex column G-10 specifically as follows: The crude protein precipitated at 70% ammonium sulfate concentration was filtered with 0.22 μM aqueous system filter membrane. 2 mL of the filtered crude protein was taken each time and added into the sephadex column G-10. Elution was performed with ultra-pure water at a flow rate of 1 mL/min for 60 min. The corresponding eluate of the 60 min elution was collected to obtain the crude protein extract. Then elution was further performed with the ultra-pure water at a flow rate of 1 mL/min for 120 min, ammonium sulfate in the sephadex column was all removed, and this part of the eluate was discarded.

(3) Cation Exchange Chromatography

5 mL of the crude protein extract obtained in the step (2) was taken, and added to a chromatographic column system (AKTA purifier 100, GE Healthcare, Sweden), and the cation exchange chromatographic column (HiPrepTMSP XL 16/10, GE Healthcare) was used. A buffer solution was obtained from 30 mM sodium acetate by adjusting pH to 4, and performing 0.22 μM suction filtration, and ultrasonic treatment for 20 min. An eluent was obtained from 30 mM sodium acetate and 1 M sodium chloride by adjusting pH to 4, and performing 0.22 suction filtration, and ultrasonic treatment for 20 min.

Specifically, that was, after 5 mL of the crude protein extract was loaded, rinsing was performed with the buffer solution at a flow rate of 1 mL/min. The use amount of the buffer solution was 40 mL. Then gradient elution was performed with a mixture of the buffer solution and the eluent. The use amount of the gradient eluent was 30 mL. The flow rate was constant at 1 mL/min.

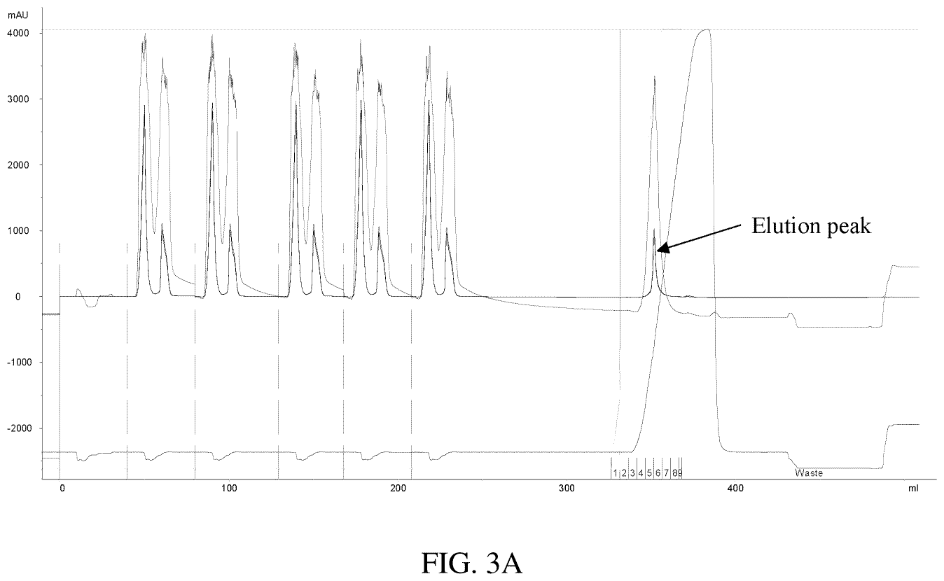

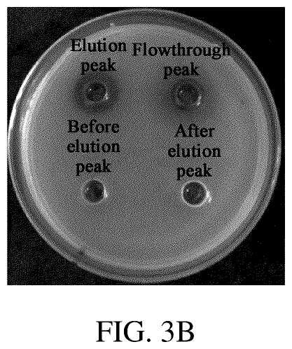

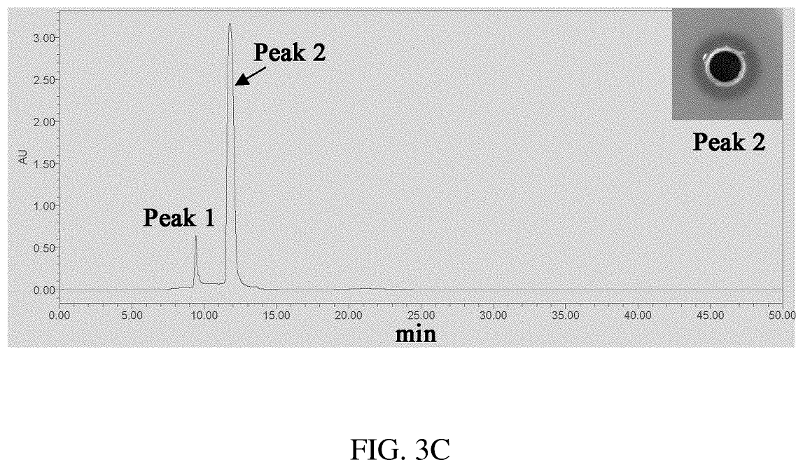

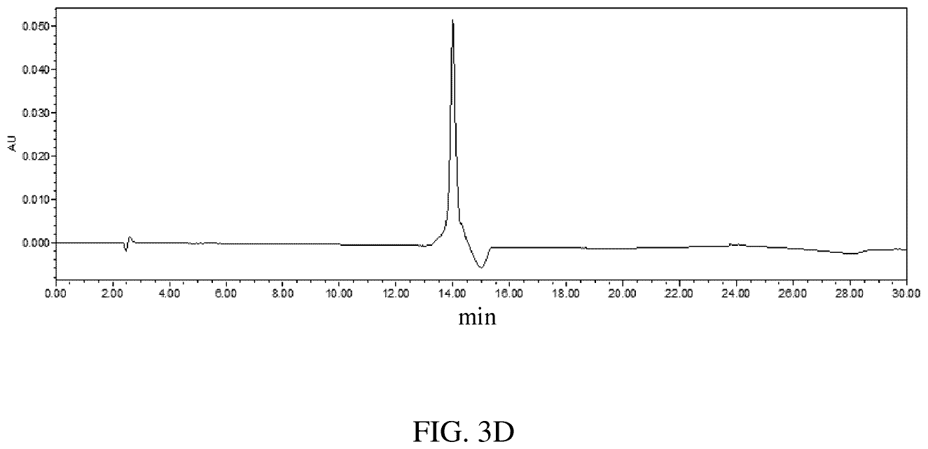

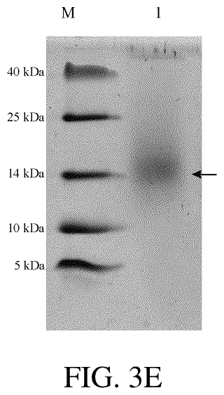

As shown in FIG. 3A, after buffer solution rinsing to a detection limit level, the concentration of sodium chloride in the gradient eluent was increased from 0 M to 1 M at a constant rate within 30 min (elution program parameters of the chromatographic column system: Gradient: 100%; time: 30 min). The flow rate was constant at 1 mL/min, and flowthrough peaks (peak appearing after the buffer solution rinsing) and elution peaks (peak appearing after eluent elution) were collected every 5 min by 5 mL/tube. After desalination was performed by using the sephadex column G-10, rotary evaporation (37° C.) was performed for concentrating to a concentration of 2 mg/mL (the concentration was determined by a BCA protein quantitative kit). As shown in FIG. 3B, the inhibitory activity of the Micrococcus luteus 10290 was detected by an Oxford cup method. The results showed that the elution peak of the 0.5 M to 1 M NaCl eluent had relatively high inhibitory activity, indicating that the bacteriocin was purified. Therefore, the eluates collected in 5th to 8th tubes were desalted by the sephadex column G-10 to obtain a protein preliminary purified solution. The following step (4) was performed.

Note: The desalination with the sephadex column G-10 can refer to the desalination of the above step (2).

(4) Reversed-Phase High-Performance Liquid Chromatography

The elution peaks of the step (3) were further purified by using preparative C18 reversed-phase high-performance liquid chromatography (high-performance liquid chromatograph waters 2998, USA). The chromatographic column was Sunfire™ Prep C18 (5 μm, 10×100 mm). The protein preliminary purified solution (a sample with inhibitory activity) obtained in the step (3) was diluted with the ultra-pure water to an appropriate concentration (that was, diluted to 0.5 to 1 mg/mL). The diluted protein preliminary purified solution was placed at 4° C. for later use after being filtered with the 0.22 μM filter membrane. An elution program was set, as shown in Table 2. An injection volume was 5 mL, the mobile phase A was the ultra-pure water (containing 0.05% TFA, v/v), and the mobile phase B was the acetonitrile (containing 0.05% TFA, v/v). The purification results are shown in FIG. 3C. It may be seen that two single peaks were obtained after purification by high performance liquid chromatography. All the single peaks were collected for the bacteriostasis experiment, which found that the peak 2 obtained by 34.5% to 37.5% mobile phase B elution had relatively high inhibitory activity.

Note: The corresponding collected eluate of the 34.5% to 37.5% mobile phase B elution was about 1 mL.

TABLE 2
Mobile Phase Elution Gradient

| | Flow | | |
|---|---|---|---|
| Time | rate | Mobile | Mobile |
| (min) | (mL/min) | phase A | phase B |
| 0 | 5 | 95 | 5 |
| 30 | 5 | 5 | 95 |
| 50 | 5 | 100 | 0 |

(5) Identification of Purity of Bacteriocin by Analytical HPLC

About 5 mg of freeze-dried bacteriocin powder was obtained after about 1 mL of corresponding collected eluate of 34.5% to 37.5% mobile phase B elution obtained in the above step 4) was freeze-dried at −80° C., and was redissolved with 5 mL of the ultra-pure water. Purity detection was performed with analytical HPLC (high-performance liquid chromatograph waters 2998, USA). The chromatographic column was Sunfire™ Prep C18 (5 μm, 4.6×250 mm). The elution program was as shown in Table 3. The mobile phase was the same as that in the above step (4), and the injection volume was 30 μL. It may be seen from FIG. 3D that a single peak appeared in the chromatogram, and the retention time was 14.13 min, indicating that the bacteriocin was well purified.

TABLE 3
Mobile Phase Elution Gradient

| | Flow | | |
|---|---|---|---|
| Time | rate | Mobile | Mobile |
| (min) | (mL/min) | phase A | phase B |
| 0 | 1 | 95 | 5 |
| 20 | 1 | 5 | 95 |
| 30 | 1 | 100 | 0 |

(6) Determination of Molecular Weight of Bacteriocin by SDS-PAGE

The above peak 2 with the relatively high antibacterial activity, purified by the preparative HPLC, underwent rotary evaporation (37° C.) for being concentrated to a concentration of 2 to 5 mg/mL (the concentration was detected by the BCA protein quantitative kit), and the molecular weight of the bacteriocin was estimated by using SDS-PAGE electrophoresis. The results were shown in FIG. 3E. There was a protein band where the molecular weight was about 14 kDa, indicating that the molecular weight of the Lactobacillus sakei ZFM225 bacteriocin was about 14 kDa.

### Embodiment 4: Inhibitory Spectrum and Determination of Minimum Inhibitory Concentration (MIC) of Lactobacillus sakei ZFM225 Bacteriocin

A total of 17 bacteria such as the gram-positive bacteria, the gram-negative bacteria and molds were selected as the indicator bacteria. Among them, the Micrococcus luteus 10209 was purchased from the China Center of Industrial Culture Collection (CICC); Staphylococcus aureus D48, Staphylococcus warneri, Staphylococcus muscae, Staphylococcus carnosus pCA 44, and Staphylococcus carnosus pet 20 were all presented by Professor Eefjan Breukink of Utrecht University in the Netherlands; Escherichia coli DH5α was purchased from the Sangon Biotechnology (Shanghai) Co., Ltd; Bacillus subtilis BAS2, Salmonella paratyphi A CMCC 50093, Salmonella paratyphi B CMCC50094, Salmonella enterica subsp. arizonae CMCC (B) 47001, Salmonella typhimurium CMCC 50015, and Candida albicans were all purchased from the National Center for Medical Culture Collections (CMCC); Pseudomonas aeruginosa ATCC 47085, Salmonella choleraesuis ATCC 13312 and Aspergillus niger ATCC 13073 were all purchased from the American Type Culture Collection (ATCC).

The inhibitory activity of the bacteriocin produced by ZFM225 was tested by an Oxford Cup inhibitory zone experiment. The specific experimental method was the same as that in the step (2) of Embodiment 1. The results are shown in Table 4, which showed that the bacteriocin produced by the Lactobacillus sakei ZFM225 was a bacteriocin with broad-spectrum inhibitory activity, which had relatively high inhibitory activity on most gram-positive bacteria, and also had certain inhibitory activity on these several gram-negative bacteria, including Escherichia coli DH5 α, Salmonella paratyphi A CMCC 50093, Salmonella choleraesuis ATCC 13312 and Pseudomonas aeruginosa ATCC 47085, but had no inhibitory effect on the molds. The bacteriocin had a relatively good inhibitory effect on some common food-borne pathogens, such as Escherichia coli, Staphylococcus aureus and Listeria monocytogenes. It was reported that the bacteriocin produced from the Lactobacillus sakei mainly inhibited growth of gram-positive pathogen Listeria. While the Lactobacillus sakei ZFM225 bacteriocin of the present disclosure had the inhibitory activity on these several gram-negative bacteria, including Escherichia coli DH5 α, Salmonella paratyphi A CMCC 50093, Salmonella choleraesuis ATCC 13312 and Pseudomonas aeruginosa ATCC 47085. Therefore, the Lactobacillus sakei ZFM225 bacteriocin of the present disclosure made up for the defect of the adverse inhibitory effect of most existing bacteriocins produced from the Lactobacillus sakei on gram-negative pathogens.

Therefore, as a biological preservative, the Lactobacillus sakei ZFM225 bacteriocin had potential application value in food safety.

TABLE 4
Inhibitory Spectrum of Bacteriocin ZFM225

| | | Inhibitory |
|---|---|---|
| | | Zone |
| | | Diameter |
| | Experimental Strain | (mm) |
| G+ | Micrococcus luteus CICC10209 | 15.7 ± 0.45 |
| | Staphylococcus aureus D48 | 13.4 ± 0.37 |
| | Staphylococcus warneri | / |
| | Staphylococcus muscae | 15.98 ± 0.56 |
| | Staphylococcus carnosus pCA 44 | 17.36 ± 0.33 |
| | Staphylococcus carnosus pet 20 | 17.56 ± 0.45 |
| G− | Escherichia coli DH5α | 13.44 ± 0.43 |
| | Bacillus subtilis BAS2 | / |
| | Salmonella paratyphi A CMCC 50093 | 11.43 ± 0.15 |
| | Salmonella paratyphi B CMCC50094 | / |
| | Salmonella enterica subsp. arizonae | / |
| | CMCC(B) 47001 | |
| | Salmonella choleraesuis ATCC 13312 | 11.97 ± 0.65 |
| | Salmonella typhimurium CMCC 50015 | / |
| | Pseudomonas aeruginosa ATCC 47085 | 13.76 ± 0.44 |
| Fungi | Candida albicans | / |
| | Aspergillus niger ATCC 13073 | / |

Note:
“/” indicates no antibacterial effect.

Taking the Micrococcus luteus 10209 and the Staphylococcus aureus D48 as the indicator bacteria, the minimum inhibitory concentration of the bacteriocin against these two indicator bacteria was determined by a 96-well titration plate method. After the indicator bacteria were inoculated into an LB liquid medium after being activated for overnight culture, the LB liquid medium was diluted until its OD₆₀₀ was 0.05 for later use.

The freeze-dried bacteriocin powder was redissolved with 0.05% acetic acid and was gradient-diluted to 2 mg/mL, 1 mg/mL, 0.5 mg/mL, 0.25 mg/mL, 0.125 mg/mL, 0.0625 mg/mL, 0.031 mg/mL, 0.015 mg/mL, 0.007 mg/mL, to obtain bacteriocin ZFM225 solutions of different concentrations. 100 μL of indicator bacteria suspension and bacteriocin diluent were added into a 96-well ELISA Plate in sequence, and underwent stationary culture at 37° C. for 24 h. Absorbance values of thalli at different bacteriocin concentrations were determined with a microplate reader at 600 nm, and a bacterial solution without the bacteriocin was taken as the control. If the OD₆₀₀ value did not increase after 24 h of culture, then the corresponding bacteriocin concentration under this condition was the minimum inhibitory concentration.

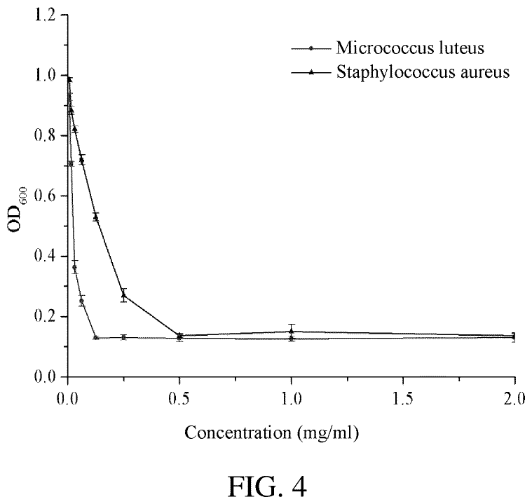

The bacteriocin was mixed with the indicator bacteria after being diluted to different concentration gradients, and the mixtures were placed at the optimum growth temperature of the indicator bacteria for 24 h. Its absorbance value at 600 nm was determined. The results were as shown in FIG. 4. It may be seen from FIG. 4 that with the increase of bacteriocin content, the growth of the two indicator bacteria was gradually decreased. The Micrococcus luteus 10209 stopped growing when the bacteriocin concentration reached 0.125 mg/mL, while for the Staphylococcus aureus D48, it was necessary for the bacteriocin concentration to reach 0.5 mg/mL. To sum up, the minimum inhibitory concentration of the bacteriocin ZFM225 against the Micrococcus luteus 10209 was 0.125 mg/mL, and the minimum inhibitory concentration against the Staphylococcus aureus D48 was 0.5 mg/mL.

### Embodiment 5: Influences of pH, Temperature and Protease on Stability of Lactobacillus sakei ZFM225 Bacteriocin

(1) Temperature Stability of Lactobacillus sakei ZFM225 Bacteriocin

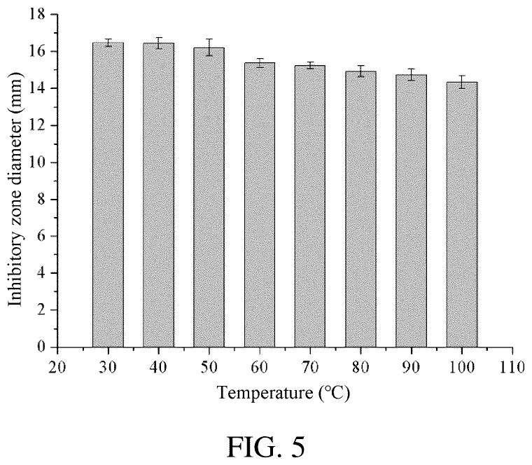

The bacteriocin ZFM225 solution (2 mg/mL) obtained in Embodiment 3 above was treated at 4° C., 37° C., 50° C., 60° C., 80° C. and 100° C. for 30 min. Taking Micrococcus luteus 10209 as the indicator bacteria, the inhibitory activity of treated bacteriocins in each group was determined with the Oxford cup method. It may be seen from FIG. 5 that in treatment at different temperatures, the inhibitory activity of the bacteriocin on the indicator bacteria basically had no change. Although the inhibitory zone diameter decreased slightly with the increase of the temperature, the retention rate of the inhibitory activity was still as high as 95% or above. It showed that the bacteriocin ZFM225 had high thermal stability.

(2) PH Stability of Lactobacillus sakei ZFM225 Bacteriocin

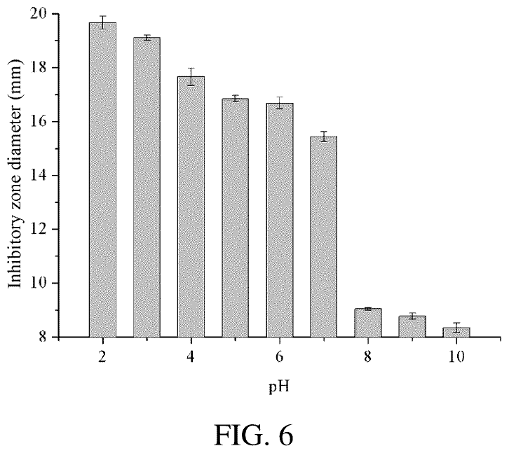

The pH of THE bacteriocin ZFM225 solution (2 mg/mL) was adjusted to 2.0, 3.0, 4.0, 5.0, 6.0, 7.0, 8.0, 9.0, 10.0 with 1 M HCl and NaOH. Taking the Micrococcus luteus 10209 as the indicator bacteria, the inhibitory activity of the bacteriocin adjusted to different pH values was detected with by the Oxford cup method. As shown in FIG. 6, the bacteriocin showed almost no loss of inhibitory activity after being treated within a range of pH 2 to 7. But, after treatment at higher pH, the inhibitory activity decreased significantly or was even lost.

(3) Enzyme Sensitivity of Lactobacillus sakei ZFM225 Bacteriocin

Enzymes used in experiments to test the sensitivity of bacteriocin to the enzymes were pepsin (pH 2.0), papain (pH 7.0), trypsin (pH 5.4) and protease K (pH 7.6). Each enzyme in a corresponding optimum pH buffer solution added with the bacteriocin to make its final concentration reach 1 mg/ml. An equal amount of freeze-dried bacteriocin powder (2 mg) was added into each portion. The mixture was placed at 37° C. for 4 h to make them fully react. Then, the fully reacted mixture was treated at 100° C. for 5 min to make the enzyme inactivated. The pH of the mixture was adjusted back to the initial pH with 1 M HCl and NaOH solution. Taking an untreated bacteriocin solution as the blank control, and taking the Micrococcus luteus 10209 as the indicator bacteria, the inhibitory activity of the bacteriocin was determined by an Oxford cup diffusion method.

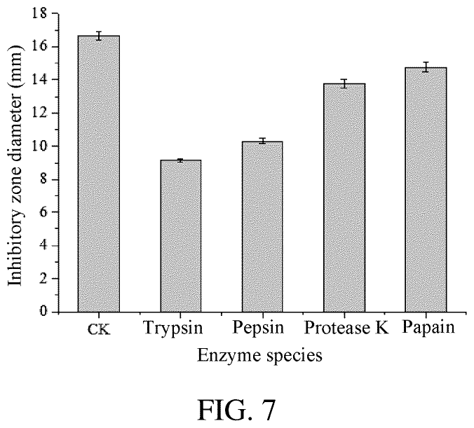

As shown in FIG. 7, the inhibitory activity of the bacteriocin decreased to a certain extent after the bacteriocin was treated with four proteases, indicating that it was a protein-like substance to play a main inhibitory effect in a bacteriocin sample. The bacteriocin was most sensitive to the trypsin and the pepsin. After being treated with these two enzymes, the activity was almost completely lost. In addition, because there are various protease substances in a human body, the sensitivity of bacteriocin samples to protease can enable the bacteriocin samples to enter the body and degraded into small molecules without adverse reactions due to enrichment in the body, which also lays a certain foundation for their future application in foods.

Finally, it should further be noted that the above enumeration is only a few specific embodiments of the present disclosure. Obviously, the present disclosure is not limited to the foregoing embodiments, and there may further be many modifications. All deformations that a person of ordinary skill in the art can directly derive or associate from the content disclosed in the present disclosure shall be considered as the protection scope of the present disclosure.

What is claimed is:

1. An isolation and purification method of a Lactobacillus sakei ZFM225 bacteriocin, comprising the steps of:
1) preparation of fermentation supernatant of Lactobacillus sakei ZFM225:
inoculating the Lactobacillus sakei ZFM225 into an MRS liquid medium for fermentation, and centrifuging obtained fermentation broth to obtain the fermentation supernatant;
2) precipitating the fermentation supernatant by an ammonium sulfate precipitation method to obtain crude protein, and desalting the crude protein to obtain crude protein extract;
3) isolating the crude protein extract by cation exchange chromatography, performing elution with a gradient eluent containing 0.5 M to 1.0 M NaCl, and desalting an obtained eluate to obtain a preliminary protein purified solution;
purifying the protein preliminary purified solution by reversed-phase high-performance liquid chromatography to obtain the Lactobacillus sakei ZFM225 bacteriocin.

2. The isolation and purification method of the Lactobacillus sakei ZFM225 bacteriocin according to claim 1, wherein in the step 1): Lactobacillus sakei ZFM225 cultured to a logarithmic growth phase is inoculated with an inoculation amount of 2% (v/v) into an MRS liquid medium with pH 6.5, and is incubated at 37° C. for 24 h; obtained fermentation broth is centrifuged to obtain fermentation supernatant.

3. The isolation and purification method of the Lactobacillus sakei ZFM225 bacteriocin according to claim 2, wherein in the step 2): ammonium sulfate powder is added to fermentation supernatant until saturation is 70±2%, stirring is performed at 4±1° C. for 10 to 14 h, and collected precipitate is centrifuged to obtain crude protein;
the crude protein is desalted by using a sephadex column G-10 to obtain crude protein extract.

4. The isolation and purification method of the Lactobacillus sakei ZFM225 bacteriocin according to claim 3, wherein in the step 3): a cation exchange chromatographic column is adopted for crude protein extract, the concentration of sodium chloride in a gradient eluent is increased from 0 M to 1 M at a constant rate within 30 min, and a corresponding eluate of 0.5 M to 1 M NaCl gradient elution is collected; desalination is performed by using a sephadex column G-10 to obtain a protein preliminary purified solution;
in gradient elution, use a mixture of a buffer solution and an eluent as a gradient eluent;
the buffer solution is obtained from 30 mM sodium acetate by adjusting pH to 4, and performing 0.22 μm suction filtration, and ultrasonic treatment for 20 min;
the eluent is obtained from 30 mM sodium acetate and 1 M sodium chloride by adjusting pH to 4, and performing 0.22 μm suction filtration and ultrasonic treatment for 20 min.

5. The isolation and purification method of the Lactobacillus sakei ZFM225 bacteriocin according to claim 4, wherein in the step 3): the protein preliminary purified solution is further purified by preparative C18 reversed-phase high-performance liquid chromatography;
TFA accounting for 0.05% of the volume content of ultra-pure water is added into the ultra-pure water to serve as a mobile phase A, and TFA accounting for 0.05% of the volume content of acetonitrile is added into the acetonitrile to serve as a mobile phase B;
Mobile Phase Elution Gradient

| | Flow | | |
|---|---|---|---|
| Time | rate | Mobile | Mobile |
| (min) | (mL/min) | phase A | phase B |
| 0 | 5 | 95 | 5 |
| 30 | 5 | 5 | 95 |
| 50 | 5 | 100 | 0 |

A corresponding collected eluate of 34.5% to 37.5% mobile phase B elution is collected to obtain a Lactobacillus sakei ZFM225 bacteriocin solution.

6. The isolation and purification method of the Lactobacillus sakei ZFM225 bacteriocin according to claim 1, wherein in the step 3): a cation exchange chromatographic column is adopted for crude protein extract, the concentration of sodium chloride in a gradient eluent is increased from 0 M to 1 M at a constant rate within 30 min, and a corresponding eluate of 0.5 M to 1 M NaCl gradient elution is collected; desalination is performed by using a sephadex column G-10 to obtain a protein preliminary purified solution;
in gradient elution, use a mixture of a buffer solution and an eluent as a gradient eluent;
the buffer solution is obtained from 30 mM sodium acetate by adjusting pH to 4, and performing 0.22 μm suction filtration, and ultrasonic treatment for 20 min;
the eluent is obtained from 30 mM sodium acetate and 1 M sodium chloride by adjusting pH to 4, and performing 0.22 μm suction filtration and ultrasonic treatment for 20 min.

7. The isolation and purification method of the Lactobacillus sakei ZFM225 bacteriocin according to claim 6, wherein in the step 3): the protein preliminary purified solution is further purified by preparative C18 reversed-phase high-performance liquid chromatography;
TFA accounting for 0.05% of the volume content of ultra-pure water is added into the ultra-pure water to serve as a mobile phase A, and TFA accounting for 0.05% of the volume content of acetonitrile is added into the acetonitrile to serve as a mobile phase B;
Mobile Phase Elution Gradient

| | Flow | | |
|---|---|---|---|
| Time | rate | Mobile | Mobile |
| (min) | (mL/min) | phase A | phase B |
| 0 | 5 | 95 | 5 |
| 30 | 5 | 5 | 95 |
| 50 | 5 | 100 | 0 |

an eluate corresponding to 34.5% to 37.5% mobile phase B elution is collected to obtain a Lactobacillus sakei ZFM225 bacteriocin solution.
